(19) United States
(12) Patent Application Publication
(10) Pub. No.: US 2015/0285816 A1
(43) Pub. Date: Oct. 8, 2015

(54) SNP FOR PREDICTING EFFECTIVENESS OF PRETERM BIRTH DIAGNOSTIC TEST

(71) Applicant: The Board of Trustees of the Leland Stanford Junior University, Stanford, CA (US)

(72) Inventors: Bruce Xuefeng Ling, Palo Alto, CA (US); Harvey J. Cohen, Los Altos, CA (US)

(21) Appl. No.: 14/676,379

(22) Filed: Apr. 1, 2015

Related U.S. Application Data
(60) Provisional application No. 61/974,114, filed on Apr. 2, 2014.

(51) Int. Cl. G01N 33/68; C12Q 1/68

(52) U.S. Cl. CPC G01N 33/689; C12Q 1/6883; C12Q 2600/156; G01N 2800/368; G01N 2800/52; C12Q 2600/118; G01N 2333/435

(57) ABSTRACT

Methods and compositions for providing a preterm birth prognosis for a pregnant individual, monitoring a pregnant individual for increased risked of preterm birth, or making a treatment decision for a pregnant individual predicting preterm birth are provided. Aspects include selecting the appropriate ITIH4 preterm peptide biomarker to detect to make a preterm birth prediction. These methods and compositions find use in providing a preterm birth prognosis for a pregnant individual.

### CROSS REFERENCE TO RELATED APPLICATIONS

Pursuant to 35 U.S.C. §119 (e), this application claims priority to the filing date of the U.S. Provisional Application Ser. No. 61/974,114 filed Apr. 2, 2014, the full disclosure of which is herein incorporated by reference.

### FIELD OF THE INVENTION

This invention pertains to the use of biomarkers in prognosing preterm birth.

### BACKGROUND OF THE INVENTION

Nearly 11% of all pregnancies in the US are result in preterm birth (<37 weeks gestation), contributing greatly to perinatal morbidity and mortality (Goldenberg, R. L. and Rouse, D. J. (1998). Prevention of premature birth. N Engl J Med 339, 313-20). Etiologies of preterm birth are largely unknown, and predictive biomarkers have yet to be adequately developed.

A decrease in the abundance of the peptide QLGLPGPPDVPDHAAYHPF (SEQ ID NO:6), a fragment of Inter-alpha-trypsin inhibitor heavy chain H4 (ITIH4), in serum has been reported to correlate with an increased likelihood of preterm births. However, the sensitivity of this biomarker as a predictive tool is relatively low: at 24 weeks, the sensitivity is 35.0% and specificity is 92.5%; at 28 weeks, the sensitivity is 65% and specificity is 82.5% (Esplin et al. Am J. Obstet Gynecol. 204(5):391.e1-e17). Thus, there is a need in the art to improve upon this method of predicting preterm birth. The present invention addresses these issues.

### SUMMARY OF THE INVENTION

Methods and compositions for providing a preterm birth prognosis for a pregnant individual, monitoring a pregnant individual for increased risked of preterm birth, or making a treatment decision for a pregnant individual predicting preterm birth are provided. Aspects include selecting the appropriate ITIH4 preterm peptide biomarker to detect to make a preterm birth prediction. These methods and compositions find use in providing a preterm birth prognosis for a pregnant individual.

### BRIEF DESCRIPTION OF THE DRAWINGS

The invention is best understood from the following detailed description when read in conjunction with the accompanying drawings. The patent or application file contains at least one drawing executed in color. Copies of this patent or patent application publication with color drawing(s) will be provided by the Office upon request and payment of the necessary fee. It is emphasized that, according to common practice, the various features of the drawings are not to-scale. On the contrary, the dimensions of the various features are arbitrarily expanded or reduced for clarity. Included in the drawings are the following figures.

FIG. 1 shows the population diversity represented by the rs2276814 SNP as taken from the NCBI SNP webpage on the world wide web, which can be accessed by placing a “www.” before “ncbi.nlm.nih.gov/SNP/snp_ref.cgi?type=rs&rs=2276814”.

FIG. 2 is an experimental design diagram of the LCMS based targeted serum peptide analysis.

FIG. 3 shows quantitative LCMS analysis to qualify the ITIH4 “L” isoform serum peptide as a preterm birth biomarker. (A) ITIH4 “L” isoform peptide chromatogram derived from preterm birth cases (gray) and healthy pregnancy controls (black). The peaks in the chromatogram were formed by the elution of “L” isoform peptides at the HPLC 16th time point, and the ionic intensities of ITIH4 “L” isoform peptide were normalized with the stable isotope labeled spiked-in ITIH4 “L*” isoform peptide. The normalized “L” isoform peptide abundances, plotted with the standard deviations, were higher in healthy pregnancy controls than in preterm birth cases. (B) Left panel: Scatter plot analysis of each subject's normalized serum abundance as a function of the baby gestational age at the time of sample collection. Left scatter plot represents preterm birth cases; right scatter plot represents healthy pregnancy controls. For either preterm birth or control sample category, bars of 75%, 50% and 25% the measures were to represent and compare the overall trend of biomarker scoring as a function of the sample classification. (B) Right panel: ROC analysis of the ITIH4 “L” isoform serum peptide as a preterm birth biomarker. 500 testing data sets, generated by bootstrapping, from the normalized abundance data were used to derive estimates of standard errors and confidence intervals for our ROC analysis. The plotted ROC curve is the vertical average of the 500 bootstrapping runs, and the box and whisker plots show the vertical spread around the average. The star denotes the cut point with the optimal sensitivity and specificity of the assay. (C) Summary of the cut point performance of sensitivity, specificity, positive predictive value, and ROC AUC. The 95% confidence intervals were listed.

FIG. 4 provides the proposed two-stage algorithm for the ITIH4 serum peptide biomarker analysis. At the first stage, the blood cell genomic DNA will be extracted for genotyping of the ITIH4 669 allele. At the second stage, either the ITIH4 “L”, or “Q”, or both isoforms will be quantified by mass spectrometric based method. Both results will be combined in the final stage to determine the preterm birth risk of the assayed subject.

FIG. 5A and FIG. 5B shows the ITIH4 L peptide abundance at different gestational ages (GA) in the serum of individuals that deliver full term birth. FIG. 5A Serum abundance normalized to total ion count. FIG. 5B Linear Regression Plot of data in FIG. 5A; p value=0.2954, indicating that normalized abundance cannot be described as a function of the gestation age.

### DETAILED DESCRIPTION OF THE INVENTION

Methods and compositions for providing a preterm birth prognosis for a pregnant individual, monitoring a pregnant individual for increased risked of preterm birth, or making a treatment decision for a pregnant individual predicting preterm birth are provided. Aspects include selecting the appropriate ITIH4 preterm peptide biomarker to detect to make a preterm birth prediction. These methods and compositions find use in providing a preterm birth prognosis for a pregnant individual. These and other objects, advantages, and features of the invention will become apparent to those persons skilled in the art upon reading the details of the compositions and methods as more fully described below.

Before the present methods and compositions are described, it is to be understood that this invention is not limited to particular method or composition described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

It must be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Methods and Compositions

Methods and compositions are provided for providing a spontaneous preterm birth prognosis for a pregnant individual, monitoring a pregnant individual for increased risk of delivering a baby preterm, and making treatment decisions for a pregnant individual. By “spontaneous preterm birth”, “preterm birth”, or “premature delivery”, it is generally meant delivery of a baby prior to full gestation, i.e. at less than 37 weeks of gestation, or three weeks or more before a baby's due date. By providing a preterm prognosis, it is generally meant to include making a prediction of the course of pregnancy and/or pregnancy outcome, and may include a prediction of the expected duration, the function, and/or a description of the course of the pregnancy. By monitoring a pregnant individual, it is meant monitoring a subject's pregnancy to detect an increased risk of delivering a baby preterm, to provide information as to the effect or efficacy of a preterm prevention therapy, etc. By making a treatment decision for a pregnant individual, e.g., a pregnant individual at risk for having a preterm birth, it is meant, for example, deciding the appropriate “treatment”, “therapy”, and the like to obtain a desired pharmacologic and/or physiologic effect, e.g. the prevention of a preterm birth. The terms “individual,” “subject,” “host,” and “patient,” are used interchangeably herein and refer to any mammalian subject for whom prognosis, diagnosis, treatment, or therapy is desired, particularly humans.

In aspects of the subject methods, the amount of preterm peptide biomarker in a biological sample from an individual is obtained, where the amount of preterm peptide biomarker is then employed to make a preterm birth prognosis for the individual. By a “peptide” it is meant an amino acid sequence of approximately 50 amino acids or less. By a “peptide biomarker” or “peptide marker” it is meant a peptide that is differentially represented, i.e. present at a different amount, in a biological sample, e.g. a blood or serum sample, from an affected individual compared to an unaffected individual. By a “preterm peptide biomarker” it is meant a peptide that is differentially represented in a biological sample, e.g. a blood or serum sample, from a pregnant individual at risk for delivering her baby preterm as compared to a pregnant individual that is not at risk for delivering her baby preterm. By the “amount”, it is generally meant the level, abundance, or concentration of preterm peptide biomarker in the biological sample.

Of particular interest in the subject methods are naturally occurring peptides derived from the ITIH4 protein. By ITIH4, it is meant the inter-alpha-trypsin inhibitor heavy chain family, member 4 protein, the full sequence of which may be found at GenBank Accession Nos. NM_{—}002218.4 (isoform 1, SEQ ID NO:1 (cDNA) and SEQ ID NO:2 (protein)) and NM_{—}001166449.1 (isoform 2, SEQ ID NO:3 (cDNA) and SEQ ID NO:4 (protein)). In some embodiments, the ITIH4 peptide of interest is an ITIH4 peptide comprising amino acid residue 669 of SEQ ID NO:2 (residue 639 of SEQ ID NO:4). In certain embodiments, the ITIH4 peptide of interest consists essentially of residues 669-687 of SEQ ID NO:2 (residues 639-657 of SEQ ID NO:4). In certain embodiments, the peptide of interest is LLGLPGPPDVPDHAAYHPF (SEQ ID NO:5). In certain embodiments, the peptide of interest is QLGLPGPPDVPDHAAYHPF (SEQ ID NO:6).

In some embodiments of the subject methods, the amount of an ITIH4 peptide comprising amino acid residue 669 of SEQ ID NO:2 (residue 639 of SEQ ID NO:4) is obtained, and a prognosis/treatment decision is made based upon the amount obtained. In some embodiments of the subject methods, the amount of ITIH4 peptide LLGLPGPPDVPDHAAYHPF (SEQ ID NO:5) is obtained, and a prognosis/treatment decision is made based upon the amount obtained. In some embodiments, the amount of peptide QLGLPGPPDVPDHAAYHPF (SEQ ID NO:6) is obtained, and a prognosis/treatment decision is made based upon the amount obtained. In some embodiments, the amount of both LLGLPGPPDVPDHAAYHPF (SEQ ID NO:5) and QLGLPGPPDVPDHAAYHPF (SEQ ID NO:6) is obtained, and a prognosis/treatment decision is made based upon the amount obtained.

### Preterm Peptide Biomarker Selection

In some instances, the ordinarily skilled artisan may select the preterm peptide biomarker to be measured to make the preterm prognosis, treatment decision, etc. In such instances, selecting which preterm peptide biomarker to detect to arrive at a preterm prognosis, treatment decision, etc., may depend on the subject individual for whom the prognosis, treatment decision, etc. is being made. As such, in some aspects of the disclosure, methods and compositions are provided for selecting an appropriate preterm peptide biomarker to measure to make a preterm birth prognosis. In other instances, no pre-selection of which preterm peptide to detect will be made; rather the amounts of both peptide biomarkers in a biological sample may be measured, and a prognosis/treatment decision may be made based upon the results of the measurements.

In aspects of the subject methods in which the artisan selects the preterm peptide biomarker to detect, the selection may be based on the genotype of the subject individual at the ITIH4 rs2276814 polymorphism. In other words, in some embodiments, an ITIH4 rs2276814 polymorphism result is obtained, and the selection of the preterm peptide biomarker is made based on the obtained polymorphism result. By an ITIH4 rs2276814 genotype, it is meant the genotype of the subject individual at the rs2276814 single nucleotide polymorphism (SNP). By the ITIH4 rs2276814 polymorphism, it is meant the single nucleotide polymorphism (SNP) that occurs at nucleotide 2065 of SEQ ID NO:1 (nucleotide 1975 of SEQ ID NO:3). The subject ITIH4 polymorphism (referred to herein as the target polymorphism) commonly appears as two variants in the population: a first variant having an A at nucleotide 2065 of SEQ ID NO:1 (referred to herein as the “A allele”) resulting in a nucleotide sequence of CAA that encodes for in a Q at residue 669 of ITIH4 (referred to herein as ITIH4 669Q variant); and a second variant having a T at nucleotide 2065 (referred to herein as the “T allele”) resulting in a nucleotide sequence CTA that encodes for a L at residue 669. In other words, the rs2276814 polymorphism may said to be an ITIH4 669Q/L polymorphism. So, for example, where a subject is genotyped for the target polymorphism, i.e. an rs2276814 polymorphism, a subject or patient sample, e.g., cells or collections thereof, e.g., a blood sample, a tissue sample, may be assayed to determine, e.g. the nucleotide sequence of the ITIH4 gene at the rs2276814 polymorphism or, e.g. the amino acid sequence encoded by the ITIH4 gene at the rs2276814 polymorphism, e.g., by using one or more genotyping reagents, such as but not limited to nucleic acid reagents such as primers etc., amplification enzymes, restriction enzymes, antibodies, buffers, etc.

In some instances, the individual will be homozygous for the A allele. In other words, AA will be detected. In such instances, the QLGLPGPPDVPDHAAYHPF (SEQ ID NO:6) peptide biomarker would be selected for detection. In other instances, the individual will be homozygous for the T allele. In other words, TT will be detected. In such instances, the LLGLPGPPDVPDHAAYHPF (SEQ ID NO:5) peptide biomarker would be selected for detection. In some instances, the individual will be heterozygous for the target polymorphism. In other words, AT will be detected. In such instances, either the QLGLPGPPDVPDHAAYHPF (SEQ ID NO:6) preterm peptide biomarker or the LLGLPGPPDVPDHAAYHPF (SEQ ID NO:5) preterm peptide biomarker, or both the QLGLPGPPDVPDHAAYHPF (SEQ ID NO:6) preterm peptide biomarker and the LLGLPGPPDVPDHAAYHPF (SEQ ID NO:5) preterm peptide biomarker, could be selected for detection.

Any convenient protocol for assaying a sample for the target polymorphism may be employed to obtain an ITIH4 rs2276814 polymorphism result. In certain embodiments, the target polymorphism will be detected at the protein level, e.g., by assaying for a polymorphic protein. In yet other embodiments, the target polymorphism will be detected at the nucleic acid level, e.g., by assaying for the presence of a nucleic acid polymorphism, e.g., the single nucleotide polymorphism (SNP) that cause expression of the polymorphic protein.

For example, polynucleotide samples derived from (e.g., obtained from) an individual may be employed. Any biological sample that comprises a polynucleotide sample comprising ITIH4 polynucleotide from the individual is suitable for use in the methods of the invention. The biological sample may be processed so as to isolate the polynucleotide. Alternatively, whole cells or other biological samples may be used without isolation of the polynucleotides contained therein. Detection of a target polymorphism in a polynucleotide sample derived from an individual can be accomplished by any means known in the art, including, but not limited to, amplification of a sequence with specific primers; determination of the nucleotide sequence of the polynucleotide sample; hybridization analysis; single strand conformational polymorphism analysis; denaturing gradient gel electrophoresis; mismatch cleavage detection; and the like. Detection of a target polymorphism can also be accomplished by detecting an alteration in the level of a mRNA transcript of the gene; aberrant modification of the corresponding gene, e.g., an aberrant methylation pattern; the presence of a non-wild-type splicing pattern of the corresponding mRNA; an alteration in the level of the corresponding polypeptide; and/or an alteration in corresponding polypeptide activity.

Detection of a target polymorphism by analyzing a polynucleotide sample can be conducted in a number of ways. A test nucleic acid sample can be amplified with primers which amplify a region known to comprise the target polymorphism(s). Genomic DNA or mRNA can be used directly. Alternatively, the region of interest can be cloned into a suitable vector and grown in sufficient quantity for analysis. The nucleic acid may be amplified by conventional techniques, such as a polymerase chain reaction (PCR), to provide sufficient amounts for analysis. The use of the polymerase chain reaction is described in a variety of publications, including, e.g., “PCR Protocols (Methods in Molecular Biology)” (2000) J. M. S. Bartlett and D. Stirling, eds, Humana Press; and “PCR Applications: Protocols for Functional Genomics” (1999) Innis, Gelfand, and Sninsky, eds., Academic Press. Once the region comprising a target polymorphism has been amplified, the target polymorphism can be detected in the PCR product by nucleotide sequencing, by SSCP analysis, or any other method known in the art. In performing SSCP analysis, the PCR product may be digested with a restriction endonuclease that recognizes a sequence within the PCR product generated by using as a template a reference sequence, but does not recognize a corresponding PCR product generated by using as a template a variant sequence by virtue of the fact that the variant sequence no longer contains a recognition site for the restriction endonuclease.

PCR may also be used to determine whether a polymorphism is present by using a primer that is specific for the polymorphism. Such methods may comprise the steps of collecting from an individual a biological sample comprising the individual's genetic material as template, optionally isolating template nucleic acid (genomic DNA, mRNA, or both) from the biological sample, contacting the template nucleic acid sample with one or more primers that specifically hybridize with a target polymorphic nucleic acid molecule under conditions such that hybridization and amplification of the template nucleic acid molecules in the sample occurs, and detecting the presence, absence, and/or relative amount of an amplification product and comparing the length to a control sample. Observation of an amplification product of the expected size is an indication that the target polymorphism contained within the target polymorphic primer is present in the test nucleic acid sample. Parameters such as hybridization conditions, polymorphic primer length, and position of the polymorphism within the polymorphic primer may be chosen such that hybridization will not occur unless a polymorphism present in the primer(s) is also present in the sample nucleic acid. Those of ordinary skill in the art are well aware of how to select and vary such parameters. See, e.g., Saiki et al. (1986) Nature 324:163; and Saiki et al (1989) Proc. Natl. Acad. Sci. USA 86:6230.

Alternatively, various methods are known in the art that utilize oligonucleotide ligation as a means of detecting polymorphisms. See, e.g., Riley et al. (1990) Nucleic Acids Res. 18:2887-2890; and Delahunty et al. (1996) Am. J. Hum. Genet. 58:1239-1246.

A detectable label may be included in an amplification reaction. Suitable labels include fluorochromes, e.g. fluorescein isothiocyanate (FITC), rhodamine, Texas Red, phycoerythrin, allophycocyanin, 6-carboxyfluorescein (6-FAM), 2′,7′-dimethoxy-4′,5′-dichloro-6-carboxyfluorescein (JOE), 6-carboxy-X-rhodamine (ROX), 6-carboxy-2′,4′,7′,4,7-hexachlorofluorescein (HEX), 5-carboxyfluorescein (5-FAM) or N,N,N′,N′-tetramethyl-6-carboxyrhodamine (TAMRA), radioactive labels, e.g. ³²P, ³⁵S, ³H; etc. The label may be a two stage system, where the amplified DNA is conjugated to biotin, haptens, etc. having a high affinity binding partner, e.g. avidin, specific antibodies, etc., where the binding partner is conjugated to a detectable label. The label may be conjugated to one or both of the primers. Alternatively, the pool of nucleotides used in the amplification is labeled, so as to incorporate the label into the amplification product.

The sample nucleic acid may be sequenced using any convenient sequencing protocol, such as a dideoxy chain termination method protocol. Genomic DNA or mRNA may be used directly. If mRNA is used, a cDNA copy may first be made. If desired, the sample nucleic acid can be amplified using a PCR. A variety of sequencing reactions known in the art can be used to directly sequence the relevant gene, or a portion thereof in which a specific polymorphism is known to occur, and detect polymorphisms by comparing the sequence of the sample nucleic acid with a reference polynucleotide that contains a target polymorphism. Any of a variety of automated sequencing procedures can be used. See, e.g., WO 94/16101; Cohen et al. (1996) Adv. Chromatography 36:127-162.

Hybridization with the variant sequence may also be used to determine the presence of a target polymorphism. Hybridization analysis can be carried out in a number of different ways, including, but not limited to Southern blots, Northern blots, dot blots, microarrays, etc. The hybridization pattern of a control and variant sequence to an array of oligonucleotide probes immobilized on a solid support, as described in U.S. Pat. No. 5,445,934, or in WO 95/35505, may also be used protocols for detecting the presence of variant sequences. Identification of a polymorphism in a nucleic acid sample can be performed by hybridizing a sample and control nucleic acids to high density arrays containing hundreds or thousands of oligonucleotide probes. Cronin et al. (1996) Human Mutation 7:244-255; and Kozal et al. (1996) Nature Med. 2:753-759.

Single strand conformational polymorphism (SSCP) analysis; denaturing gradient gel electrophoresis (DGGE); mismatch cleavage detection; and heteroduplex analysis in gel matrices can also be used to detect polymorphisms. Alternatively, where a polymorphism creates or destroys a recognition site for a restriction endonuclease (restriction fragment length polymorphism, RFLP), the sample is digested with that endonuclease, and the products size fractionated to determine whether the fragment was digested. Fractionation is performed by gel or capillary electrophoresis, particularly acrylamide or agarose gels. The aforementioned techniques are well known in the art. Detailed description of these techniques can be found in a variety of publications, including, e.g., “Laboratory Methods for the Detection of Mutations and Polymorphisms in DNA” (1997) G. R. Taylor, ed., CRC Press, and references cited therein.

Screening for the target polymorphism may be performed at the protein level. In other words, the target polymorphism may be detected by analyzing a polypeptide sample from the subject individual. In such cases, the detection of the target polymorphism may be based on the functional or antigenic characteristics of the protein. Protein truncation assays are useful in detecting deletions that may affect the biological activity of the protein. Various immunoassays designed to detect polymorphisms in polymorphic polypeptides may be used in screening. As another example, functional protein assays may be employed. For example, the activity of the encoded a polymorphic polypeptide may be determined by comparison with a reference polypeptide lacking a specific polymorphism.

Screening for the target polymorphism may be performed at the peptide level. In other words, the target polymorphism may be detected by analyzing the peptide content of a biological sample from the subject individual by mass spectrometry. As demonstrated in the working examples below, the subject preterm peptide biomarkers of interest have characteristic molecular weight, m/z peak, and Rf values that are different from one another. Accordingly, the genotype of the individual may be determined by analyzing the biological sample for the presence of peptide QLGLPGPPDVPDHAAYHPF (SEQ ID NO:6) and peptide LLGLPGPPDVPDHAAYHPF (SEQ ID NO:5).

Additional references describing various protocols for detecting the presence of a target polymorphism include, but are not limited to, those described in: U.S. Pat. Nos. 6,703,228; 6,692,909; 6,670,464; 6,660,476; 6,653,079; 6,632,606; 6,573,049; the disclosures of which are herein incorporated by reference.

In some embodiments, the selection of which preterm peptide biomarker to detect is made based on a demographic survey of the ethnicity of the subject individual. As discussed in the working example below, the “T” allele at nucleotide 2065, which encodes the ITIH4 669L variant, is found with greater frequency in Caucasian and Hispanic populations than in African American populations. In contrast, the “A” allele at nucleotide 2065, which encodes an ITIH4 669Q variant, is found with greater frequency in African American populations than in Caucasian and Hispanic populations. As such, if the subject individual was determined to be of Caucasian or Hispanic descent, the LLGLPGPPDVPDHAAYHPF (SEQ ID NO:5) peptide biomarker would be selected for use in prognosing preterm birth risk, whereas if the subject individual was determined to be African American descent, the QLGLPGPPDVPDHAAYHPF (SEQ ID NO:6) peptide biomarker would be selected for use in prognosing preterm birth risk.

In some embodiments, the selection of which preterm peptide biomarker to detect is based upon both the ITIH4 rs2276814 polymorphism result of the subject individual and the demographic survey of the subject individual.

In some instances, a first artisan may determine which preterm peptide biomarker to detect, and provide the information to a second artisan or a patient, e.g. in the form of a report. Thus, for example, in some embodiments, the subject methods comprise obtaining a biological sample, detecting the ITIH4 rs2276814 genotype in the biological sample, and providing, i.e. generating or outputting, a report that includes the results of an evaluation of the rs2276814 polymorphism in a biological sample, which report can be provided in the form of an electronic medium (e.g., an electronic display on a computer monitor), or in the form of a tangible medium (e.g., a report printed on paper or other tangible medium). Any form of report may be provided, e.g. as known in the art or as described in greater detail below.

### Measuring Preterm Peptide Biomarker Abundance

As discussed above, the ordinarily skilled artisan may wish to proceed without selecting which preterm peptide biomarker to detect, i.e. without obtaining an rs2276814 polymorphism or a demongraphic survey, by, for example, measuring the abundance of both subject preterm peptide biomarkers in a biological sample from an individual. Alternatively, the ordinarily skilled artisan may determine the appropriate preterm peptide biomarker to detect, and obtain the amount of the determined preterm peptide biomarker in the biological sample. In either case, in practicing aspects of the subject methods, the amount of preterm peptide biomarker in a biological sample from the individual is obtained, where the amount of preterm peptide biomarker in the biological sample is then employed to make a preterm birth prognosis for the individual.

The term “biological sample” encompasses a variety of sample types obtained from an organism and can be used in a diagnostic or monitoring assay. The term encompasses blood and other liquid samples of biological origin or cells derived therefrom and the progeny thereof. The term encompasses samples that have been manipulated in any way after their procurement, such as by treatment with reagents, solubilization, or enrichment for certain components. The term encompasses a clinical sample, and also includes cell supernatants, cell lysates, serum, plasma, biological fluids, and tissue samples.

Clinical samples for use in the subject methods may be obtained from a variety of sources, particularly blood samples. Once a sample is obtained, it can be used directly, frozen, or maintained in appropriate culture medium for short periods of time. Typically the samples will be from human patients, although animal models may find use, e.g. equine, bovine, porcine, canine, feline, rodent, e.g. mice, rats, hamster, primate, etc. Any convenient tissue sample that demonstrates the differential representation in a patient of the subject peptide biomarker selected as described herein may be evaluated in the subject methods. Typically, a suitable sample source will be derived from fluids into which the subject peptide biomarker has been released. Sample sources of particular interest include blood samples or preparations thereof, e.g., whole blood, or serum or plasma. A sample volume of blood, serum, or urine between about 2 μl to about 2,000 μl is typically sufficient for determining the level of a preterm peptide biomarker. Generally, the sample volume will range from about 10 μl to about 1,750 μl, from about 20 μl to about 1,500 μl, from about 40 μl to about 1,250 μl, from about 60 μl to about 1,000 μl, from about 100 μl to about 900 μl, from about 200 μl to about 800 μl, from about 400 μl to about 600 μl. In many embodiments, a suitable initial source for the human sample is a blood sample. As such, the sample employed in the subject assays is generally a blood-derived sample. The blood derived sample may be derived from whole blood or a fraction thereof, e.g., serum, plasma, etc., where in some embodiments the sample is derived from blood, allowed to clot, and the serum separated and collected to be used to assay.

The subject sample may be treated in a variety of ways so as to enhance detection of the subject peptide. For example, where the sample is blood, the red blood cells may be removed from the sample (e.g., by centrifugation) prior to assaying. Such a treatment may serve to reduce the non-specific background levels of detecting the level of the subject peptide. As another example, the sample may be purified by removing proteins, nucleic acids, and the like, e.g. by liquid chromatography, e.g. HPLC, to obtain a sample that is substantially pure in naturally occurring peptides. Detection of a subject peptide may also be enhanced by concentrating the sample using procedures well known in the art (e.g. acid precipitation, alcohol precipitation, salt precipitation, hydrophobic precipitation, filtration (using a filter which is capable of retaining molecules greater than 30 kD, e.g. Centrim 30™), affinity purification). In some embodiments, the pH of the test and control samples will be adjusted to, and maintained at, a pH which approximates neutrality (i.e. pH 6.5-8.0). Such a pH adjustment will prevent complex formation, thereby providing a more accurate quantitation of the level of marker in the sample. In some embodiments, e.g. where the sample is a urine sample, the pH of the sample is adjusted and the sample is concentrated in order to enhance the detection of the marker.

The subject biological sample is typically obtained from the individual during the second or third trimester of gestation. By “gestation” it is meant the duration of pregnancy in a mammal, i.e. the period of development in the uterus from conception until birth. The time interval of a gestation plus two weeks, i.e. to the last menstrual period, is called the gestation period. Human gestation can be divided into three trimesters, each three months long. The first trimester is from the last menstrual period to the 13th week, the second trimester is from the 14th to 27th week, and the third trimester is from the 28th week to 42 weeks. A subject sample may be obtained, on or before 37 weeks of gestation, e.g., on or before week 30 of gestation, on or before week 25 of gestation, but typically no earlier than about week 20 of gestation. For example, the subject sample may be obtained at weeks 20-25 of gestation, at weeks 26-30 of gestation, at weeks 31-34 weeks of gestation, at weeks 35-37 of gestation, for example, week 20, week 21, week 22, week 23, week 24, week 25, week 26, week 27, week 28, week 29, week 30, week 31, week 32, week 33, week 34, week 35, or week 36 of gestation.

In certain embodiments, the sample is a serum or serum-derived sample. Any convenient methodology for producing a fluid serum sample may be employed. In many embodiments, the method employs drawing venous blood by skin puncture (e.g., finger stick, venipuncture) into a clotting or serum separator tube, allowing the blood to clot, and centrifuging the serum away from the clotted blood. The serum is then collected and stored until assayed. In some instances, the obtaining comprises drawing the sample from the subject. In other instances, the obtaining comprises receiving a sample from a practitioner, where the practitioner has drawn the sample from the individual. Once the patient derived sample is obtained, the sample is assayed to detect the level of the subject peptide in the sample.

The amount of the subject preterm peptide biomarker in the biological sample may be detected by any convenient method for detecting peptide in a biological sample. For example, Mass Spectrometry (MS) may be employed. In MS, a sample (which may be solid, liquid, or gas) is ionized; the ions are separated according to their mass-to-charge ratio, e.g. by magnetic sector, by radio frequencies (RF) quadrupole field, by time of flight (TOF), etc.; the ions are dynamically detected by some mechanism capable of detecting energetic charged particles, and the signal is processed into the spectra of the masses of the particles of that sample. In some instances, tandem mass spectrometry (MS/MS or MS²) may be employed, for example, to determine the sequences of the peptides separated by MS. For example, a first mass analyzer isolates one peptide from many entering a mass spectrometer. A second mass analyzer then stabilizes the peptide ions and promotes their fragmentation, e.g. by collision-induced dissociation (CID), electron capture dissociation (ECD), electron transfer dissociation (ETD), infrared multiphoton dissociation (IRMPD), blackbody infrared radiative dissociation (BIRD), electron-detachment dissociation (EDD), surface-induced dissociation (SID), etc. A third mass analyzer then sorts the fragments produced from the peptides. For example, a sample may be applied to an LTQ ion trap mass spectrometer equipped with a Fortis tip mounted nano-electrospray ion source, and the fraction scanned with a mass range of 400-2000 m/z. This first MS scan is followed by two data-dependent scans of the two most abundant ions observed in the first full MS scan. Tandem MS can also be done in a single mass analyzer over time, as in a quadrupole ion trap. In some instances, MS is combined with other technologies, e.g. multiple reaction monitoring (MRM) is coupled with stable isotope dilution (SAD) mass spectrometry (MS), which allowed quantitative assays for peptides to be performed with minimum restrictions and the ease of assembling multiple peptide detections in a single measurement. Other methods for detecting peptides in a sample by MS and measuring the abundance of peptides in a sample are well known in the art; see, e.g. the teachings in US 2010/0163721, the full disclosure of which is incorporated herein by reference. As demonstrated in the working examples herein, the subject preterm peptide biomarker LLGLPGPPDVPDHAAYHPF (SEQ ID NO:5) can be readily differentiated from the subject preterm peptide biomarker QLGLPGPPDVPDHAAYHPF (SEQ ID NO:6) using methods known in the art. For example, the peptides may be differentiated from one another by mass spectrometry, e.g. by liquid chromatography followed by mass spectrometry, the LLGLPGPPDVPDHAAYHPF (SEQ ID NO:5) peptide having a molecular weight of about 2013, an m/z (mass to charge ratio) of 671 with charge (z) of +3, and a retention time of 31.4 min (sd: 0.42) on a 60 min C18 HPLC column, versus the QLGLPGPPDVPDHAAYHPF (SEQ ID NO:6) peptide having a molecular weight of about 2027, an m/z of 677 with a charge (z) of +3, and a retention time of about 31 minutes on a 60 min C18 HPLC column.

Alternatively, non-MS based-methods for measuring the amount of peptides in a sample may be employed. These include, for example, immune-based methods such as ELISA, western blotting, flow cytometry, immunohistochemistry, and the like. In such methods, antibodies or other protein detection reagents that are specific for the subject peptide are used to detect the peptide and its abundance. In some instances, it may be desirable that the protein detection reagents, e.g., antibodies, are specific for the subject peptide and do not react with the full-length ITIH4 polypeptide. Typically, such antibodies will be specific for a domain created by the cleavage event that generated the peptide, i.e., the antibodies will be cleavage site-specific antibodies. Antibodies that are specific to the polypeptide(s) and not the peptide marker(s) may also be used, which serve as negative control(s).

The resultant data provides information regarding the amount in the sample of the selected preterm peptide biomarker, wherein the information is in terms of whether or not the peptide is present and, typically, at what level, and wherein the data may be both qualitative and quantitative. As such, where detection is qualitative, the methods provide a reading or evaluation, e.g., assessment, of whether or not the subject peptide is present in the sample being assayed. In yet other embodiments, the methods provide a quantitative detection of whether the subject peptide is present in the sample being assayed, i.e., an evaluation or assessment of the actual or relative amount of the subject peptide in the sample being assayed. In such embodiments, the quantitative detection may be absolute or, if the method is a method of detecting two or more different peptides in a sample, relative. As such, the term “quantifying” when used in the context of quantifying a target peptide in a sample can refer to absolute or to relative quantification. Absolute quantification may be accomplished by inclusion of known concentration(s) of one or more control peptide(s) and referencing the detected level of the target peptide(s) with the known control peptide(s) (e.g., through generation of a standard curve). Alternatively, relative quantification can be accomplished by comparison of detected levels or amounts between two or more different target peptide(s) to provide a relative quantification of each of the two or more different peptide(s), e.g., relative to each other.

For example, as demonstrated in the working examples herein, a reference peptide LLGLPGPPDVP*DHAAYHPF (SEQ ID NO:5) having a molecular weight of about 2044 may be employed to quantify the amount of LLGLPGPPDVPDHAAYHPF (SEQ ID NO:5) peptide in a biological sample, the reference peptide comprising a Pro(u-13C5, 15N) at residue 11 (designated P*). Similarly, a reference peptide QLGLPGPPDVP*DHAAYHPF (SEQ ID NO:6) having a molecular weight of about 2059 may be employed to quantify the amount of QLGLPGPPDVPDHAAYHPF (SEQ ID NO:6) peptide in a biological sample, the reference peptide comprising a Pro(u-13C5, 15N) at residue 11 (again, designated as P*). The amount of LLGLPGPPDVPDHAAYHPF (SEQ ID NO:5) can then be expressed as the normalized amount of the measured amount of LLGLPGPPDVPDHAAYHPF (SEQ ID NO:5) relative to the measured amount of LLGLPGPPDVP*DHAAYHPF (SEQ ID NO:5) in the sample, while the amount of QLGLPGPPDVPDHAAYHPF (SEQ ID NO:6) can then be expressed as the normalized amount of the measured amount of QLGLPGPPDVPDHAAYHPF (SEQ ID NO:6) relative to the measured amount of QLGLPGPPDVP*DHAAYHPF (SEQ ID NO:6) in the sample.

In some instances, only the amount of the subject preterm peptide biomarker(s), i.e. LLGLPGPPDVPDHAAYHPF (SEQ ID NO:5), QLGLPGPPDVPDHAAYHPF (SEQ ID NO:6), or LLGLPGPPDVPDHAAYHPF (SEQ ID NO:5) and QLGLPGPPDVPDHAAYHPF (SEQ ID NO:6), will be quantified in the sample, and the amount used to make a preterm prognosis, treatment decision, etc. In other instances, the amounts of one or more other preterm biomarkers will be measured as well, and the amounts of the subject preterm peptide biomarker(s) used in combination with the amounts of these other preterm peptide biomarkers to arrive at a preterm prognosis, treatment decision, etc. Example of other preterm peptide biomarkers know in the art include NVHSAGAAGSRMNFRPGVLSSRQLGLPGPPDVPDHAAYHPF (SEQ ID NO:7), having a molecular weight of about 4280, NVHSAGAAGSRM(O)NFRPGVLSSRQLGLPGPPDVPDHAAYHPF (SEQ ID NO:7), having a molecular weight of about 4295 (where M(O) represents an oxidized methionine), and NVHSGSTFFKYYLQGAKIPKPEASFSPR (SEQ ID NO:8), having a molecular weight of about 3970, as disclosed in US Application No. 2010/0297679. These and other preterm peptide biomarkers may be used in combination with the preterm peptide biomarkers of the present disclosure by any number of methods in the art. For example, the absolute or relative amount of each additional peptide biomarker in the biological sample may be considered independently to arrive at a profile for the biological sample. Alternatively, the amounts of each additional peptide biomarker may be considered in combination with the subject peptide markers, using, for example, weighting to arrive at a single preterm peptide score for the biological sample.

In some instances, the subject methods further comprise providing the results of the preterm birth peptide(s) quantification in a biological sample as a report. In other words, the subject methods comprise obtaining a biological sample, detecting the abundance of peptide(s) for a preterm peptide or panel of preterm peptides in the biological sample, evaluating the detected abundance of the subject peptide(s) in the sample, and providing, i.e. generating, a report that includes the evaluated levels of the subject preterm peptide(s). Thus, a subject method may further include a step of generating or outputting a report providing the results of an evaluation of the abundance of preterm peptide(s) in a biological sample, which report can be provided in the form of an electronic medium (e.g., an electronic display on a computer monitor), or in the form of a tangible medium (e.g., a report printed on paper or other tangible medium). Any form of report may be provided, e.g. as known in the art or as described in greater detail below.

### Reports

A “report,” as described herein, is an electronic or tangible document which includes report elements that provide information of interest relating to a subject monitoring assessment and its results. In some embodiments, a subject report includes the results of one or more aspects of the subject methods directed to obtaining the amount of preterm peptide biomarker in a biological sample, e.g. an ITIH4 rs2276814 polymorphism result, a demographic survey result, a peptide abundance result, etc., as discussed in greater detail above. In some embodiments, a subject report includes at least a preterm birth prediction, i.e. a prediction as to the likelihood of a patient developing preterm, e.g. as an aspect of the subject methods directed to providing a preterm prognosis for an individual, discussed in greater detail above. A subject report can be completely or partially electronically generated. A subject report can further include one or more of: 1) information regarding the testing facility; 2) service provider information; 3) patient data; 4) sample data; 5) an assessment report, which can include various information including: a) reference values employed, and b) test data, where test data can include, e.g., an ITIH4 rs2276814 polymorphism result, a demographic survey result, a peptide abundance result; and 6) other features.

The report may include information about the testing facility, which information is relevant to the hospital, clinic, or laboratory in which sample gathering and/or data generation was conducted. Sample gathering can include obtaining a fluid sample, e.g. blood, saliva, urine etc.; a tissue sample, e.g. a tissue biopsy, etc. from a subject. Data generation can include rs2276814 genotype, the absolute or relative concentrations of subject preterm peptide(s) in the sample, etc. This information can include one or more details relating to, for example, the name and location of the testing facility, the identity of the lab technician who conducted the assay and/or who entered the input data, the date and time the assay was conducted and/or analyzed, the location where the sample and/or result data is stored, the lot number of the reagents (e.g., kit, etc.) used in the assay, and the like. Report fields with this information can generally be populated using information provided by the user.

The report may include information about the service provider, which may be located outside the healthcare facility at which the user is located, or within the healthcare facility. Examples of such information can include the name and location of the service provider, the name of the reviewer, and where necessary or desired the name of the individual who conducted sample gathering and/or data generation. Report fields with this information can generally be populated using data entered by the user, which can be selected from among pre-scripted selections (e.g., using a drop-down menu). Other service provider information in the report can include contact information for technical information about the result and/or about the interpretive report.

The report may include a patient data section, including patient medical history (which can include, e.g., age, race, serotype, prior preterm births experienced by the patient, chronic health problems in the patient, such as high blood pressure, diabetes, and clotting disorders, and any other characteristics of the pregnancy, e.g. carrying more than one baby, problems with the uterus or cervix, infections that have occurred during pregnancy, cigarette smoking, alcohol use, or illicit drug use during pregnancy), as well as administrative patient data such as information to identify the patient (e.g., name, patient date of birth (DOB), gender, mailing and/or residence address, medical record number (MRN), room and/or bed number in a healthcare facility), insurance information, and the like), the name of the patient's physician or other health professional who ordered the monitoring assessment and, if different from the ordering physician, the name of a staff physician who is responsible for the patient's care (e.g., primary care physician).

The report may include a sample data section, which may provide information about the biological sample analyzed in the monitoring assessment, such as the source of biological sample obtained from the patient (e.g. blood, saliva, or type of tissue, etc.), how the sample was handled (e.g. storage temperature, preparatory protocols) and the date and time collected. Report fields with this information can generally be populated using data entered by the user, some of which may be provided as pre-scripted selections (e.g., using a drop-down menu).

The report may include an assessment report section, which may include information generated after processing of the data as described herein. The interpretive report can include values associated with one or more reference samples. The interpretive report can include a prediction of the likelihood that the subject will give birth prematurely. The interpretive report can include, for example, the results of an rs2276814 genotyping, e.g. “AA”, “AT”, or “TT”; the results of a peptide detection assay (e.g., “1.5 nmol/liter LLGLPGPPDVPDHAAYHPF (SEQ ID NO:5) in serum” or “1.5 nmol/liter QLGLPGPPDVPDHAAYHPF (SEQ ID NO:6) in serum”); an evaluation of the results of the peptide detection assay (e.g. “a preterm birth score of 0.2”) and interpretation, i.e. prediction, diagnosis, or characterization. The assessment portion of the report can optionally also include a recommendation(s). For example, where the results indicate that preterm is likely, the recommendation can include a recommendation to receive 17 alpha-hydroxyprogesterone caproate injections or use a progesterone-based gel, etc., as recommended in the art.

It will also be readily appreciated that the reports can include additional elements or modified elements. For example, where electronic, the report can contain hyperlinks which point to internal or external databases which provide more detailed information about selected elements of the report. For example, the patient data element of the report can include a hyperlink to an electronic patient record, or a site for accessing such a patient record, which patient record is maintained in a confidential database. This latter embodiment may be of interest in an in-hospital system or in-clinic setting. When in electronic format, the report is recorded on a suitable physical medium, such as a computer readable medium, e.g., in a computer memory, zip drive, CD, DVD, etc.

It will be readily appreciated that the report can include all or some of the elements above, with the proviso that the report generally includes at least the elements sufficient to provide the analysis requested by the user (e.g. prediction or prognosis with regard to the likelihood of preterm birth).

### Utility

As discussed above, preterm peptide abundance results find a number of uses in the clinic, including, for example, for providing a spontaneous preterm birth prognosis for a pregnant individual, monitoring a pregnant individual for increased risk of delivering a baby preterm, and making treatment decisions for a pregnant individual. By “spontaneous preterm birth”, “preterm birth”, or “premature delivery”, it is generally meant delivery of a baby prior to full gestation, i.e. at less than 37 weeks of gestation, i.e. three weeks or more before a baby's due date. The terms “individual,” “subject,” “host,” and “patient,” are used interchangeably herein and refer to any mammalian subject for whom prognosis, diagnosis, treatment, or therapy is desired, particularly humans.

By providing a preterm prognosis, it is generally meant to include making a prediction of the course of pregnancy and/or pregnancy outcome, and may include a prediction of the expected duration, the function, and/or a description of the course of the pregnancy. The prediction may made about 1 week or more in advance of delivery, e.g. 2 weeks, 3 weeks, 4 weeks, 5 weeks, 6 weeks or more, for example, 7 weeks, 8 weeks, 9 weeks, or 10 weeks or more, in some instances 11 weeks or more, 12 weeks or more, 13 weeks or more, 14 weeks or more, or about 15 weeks. For example, the subject methods and compositions may be used to make a prediction as to the course of the pregnancy in about weeks 20-35 of gestation, for example, at about week 20, week 21, week 22, week 23, week 24, week 25, week 26, week 27, week 28, week 29, week 30, week 31, week 32, week 34, week 35, or week 36 of gestation. In some instances, the prognosis will include a risk assessment as to the likelihood that a pregnant individual will have a preterm delivery, and/or when the preterm delivery will occur. In some instances, the risk assessment will include providing a risk classification for the individual, e.g. a level of risk (or likelihood) that a subject will deliver a baby preterm. A subject may be classified into a risk group or classified at a level of risk based on the methods of the present disclosure, e.g. high, medium, or low risk, where a “risk group” is a group of subjects or individuals with a similar level of risk for a particular clinical outcome.

By monitoring a pregnant individual, it is meant monitoring a subject's pregnancy to detect an increased risk of delivering a baby preterm, to provide information as to the effect or efficacy of a preterm prevention therapy, and the like. For example, the pregnant individual may be monitored daily, weekly, biweekly, etc., where a reduction in the subject peptide biomarker abundance in a biological sample at a later monitoring session as compared to an earlier monitoring session indicates an increased likelihood that the individual will deliver prematurely.

By making a treatment decision for a pregnant individual, e.g., a pregnant individual at risk for having a preterm birth, it is meant, for example, deciding the appropriate “treatment”, “therapy”, and the like to obtain a desired pharmacologic and/or physiologic effect, e.g. the prevention of a preterm birth. “Treatment” as used herein covers any treatment of a condition in a mammal, and includes: (a) preventing the condition from occurring in a subject which may be predisposed to the condition but has not yet been diagnosed as having it; (b) inhibiting the condition, i.e., arresting its development; or (c) relieving the condition, i.e., causing regression of the condition. The therapeutic agent may be administered before, during or after the onset of condition. The treatment of ongoing condition, where the treatment stabilizes or reduces the undesirable clinical symptoms of the patient, e.g. a heightened risk of giving birth prematurely, is of particular interest. Such treatment is desirably administered upon the determination of a risk in having a preterm birth. Typically the effect will be prophylactic in terms of completely or partially preventing a preterm birth, e.g. delaying a preterm birth one or more days, e.g. 2, 3, 4, 5, 6, 7 or more days, for example 2 weeks, 3 weeks, 4 weeks or more, in some instances 5 weeks, 6 weeks, 7 weeks, 8 weeks, 9 weeks, 10 weeks or more.

In some embodiments, the preterm peptide abundance result is employed by comparing it to a reference, to identify similarities or differences with the reference, where the similarities or differences that are identified are then employed to predict if a pregnant woman will give birth prematurely, to monitor the pregnant woman for an increased likelihood of giving birth prematurely, etc. For example, a reference may be a concentration of a reference peptide, e.g. LLGLPGPPDVP*DHAAYHPF (SEQ ID NO:5), QLGLPGPPDVP*DHAAYHPF(SEQ ID NO:6), as described above, that is representative of individuals that give birth prematurely (i.e. a positive control) or that deliver at full term (i.e. a negative control) which may be added to a patient sample and used as an internal reference/control in the evaluation of the preterm peptide abundance result for a given patient. As another example, a reference may be a preterm peptide abundance result that is representative of individuals that give birth prematurely (i.e. a positive control) or that deliver at full term (i.e. a negative control), which may be used, for example, as a reference/control in the evaluation of the preterm peptide abundance result for a given patient. References are preferably the same type of sample or, if peptide abundance results, are based on the evaluation of the same type of sample as the sample that was employed to generate the preterm peptide abundance result for the individual being assessed. For example, if the serum of an individual is being evaluated, the reference/control would preferably be of serum.

In certain embodiments, the obtained preterm peptide abundance result is compared to two or more references. For example, the obtained preterm peptide abundance result may be compared to a negative reference and a positive reference to obtain confirmed information regarding if the individual will deliver a baby prematurely. The comparison step results in information regarding how similar or dissimilar the obtained preterm peptide abundance result is to the control/reference, which similarity/dissimilarity information is employed to prognose preterm birth, for example to predict the onset of a preterm birth, monitor a patient, make a treatment decision, etc. Similarity may be based on relative peptide abundance, absolute peptide abundance or a combination of both. In certain embodiments, a similarity determination is made using a computer having a program stored thereon that is designed to receive input for a preterm peptide abundance result obtained from a subject, e.g., from a user, determine similarity to one or more reference peptide representations, and return a preterm birth prognosis, e.g., to a user (e.g., lab technician, physician, pregnant individual, etc.).

In other embodiments, the preterm birth score is employed directly, i.e. without comparison to a reference, to make a prediction, diagnosis, or characterization.

Thus, for example, as demonstrated in the working examples below, a 2.5-fold reduction or more in the amount of LLGLPGPPDVPDHAAYHPF (SEQ ID NO:5) or QLGLPGPPDVPDHAAYHPF (SEQ ID NO:6) in a patient sample at about 24 weeks of gestation relative to the amount of peptide in a same-staged unaffected individual, i.e. a pregnant individual that will give birth at full term, is predictive of about a 70% risk of preterm birth.

In some instances, the method further comprises detecting one or more clinical parameter, and providing a prognosis based on the preterm birth score and these one or more clinical parameters. Clinical parameters that suggest an increased risk of delivering preterm include prior preterm births by the patient; chronic health problems in the patient, such as high blood pressure, diabetes, and clotting disorders; carrying more than one baby; problems with the uterus or cervix; infections that have occurred during pregnancy; and cigarette smoking, alcohol use, or illicit drug use during pregnancy. Thus, in some instances, the method further comprises measuring one or more clinical parameters selected from prior preterm births by the patient; the presence of high blood pressure, diabetes, and/or clotting disorders; carrying more than one baby; problems with the uterus or cervix; infections that have occurred during pregnancy; and patient habits such as cigarette smoking, alcohol use, or illicit drug use during pregnancy; and employing those one or more clinical parameters in combination with the subject preterm peptide abundance result to make a preterm prediction.

In some embodiments, the preterm birth prognosis may be provided by providing, i.e. generating, a written report that includes the practitioner's monitoring assessment, i.e. the practitioner's prediction regarding the increased likelihood of preterm birth (a “preterm prediction”). Thus, a subject method may further include a step of generating or outputting a report providing the results of a monitoring assessment, which report can be provided in the form of an electronic medium (e.g., an electronic display on a computer monitor), or in the form of a tangible medium (e.g., a report printed on paper or other tangible medium). Any form of report may be provided, e.g. as known in the art or as described in greater detail herein.

As demonstrated herein, a prognostic test for preterm birth that relies upon detecting a decreased abundance of preterm peptide biomarker QLGLPGPPDVPDHAAYHPF (SEQ ID NO:6) may yield a false positive result if the genome of the individual encodes for the LLGLPGPPDVPDHAAYHPF (SEQ ID NO:5) variant. As such, following the subject methods, a preterm birth prognosis by detecting the preterm peptide biomarker LLGLPGPPDVPDHAAYHPF (SEQ ID NO:5) variant will provide a preterm prognosis with greater accuracy than methods currently used in the art.

In some instances, the measurement of the subject preterm peptide biomarkers disclosed herein provides for a preterm prognosis that has an improved specificity, sensitivity, and accuracy over a preterm prognosis or diagnosis made using standard methods known in the art. By sensitivity, also called the “recall rate” in some fields, it is meant the proportion of actual positives which are correctly identified as such (e.g. the percentage of individuals at risk for developing preeclampsia that really are at risk for developing preeclampsia). By specificity, it is meant the proportion of actual negatives which are correctly identified as such (e.g. the percentage of healthy people that are correctly identified as not being at risk for developing preeclampsia). By accuracy, it is meant the degree of closeness of measurements of a quantity to that quantity's true value (e.g. the percentage of true results overall that are correctly called, i.e. the percentage of individuals at risk for developing preeclampsia that accurately identified plus the percentage of healthy individuals that accurately identified). Mathematically, these terms may be defined as follows:

$$Sensitivity = \frac{( Number of true positives )}{( Number of true positives + Number of false negatives )}$$

$$Specificity = \frac{( Number of true negatives )}{( Number of true negatives + Number of false positives )}$$

$$Accuracy = \frac{( Number of true positives + true negatives )}{( Number of true positives + false positives + false negatives + true negatives )}$$

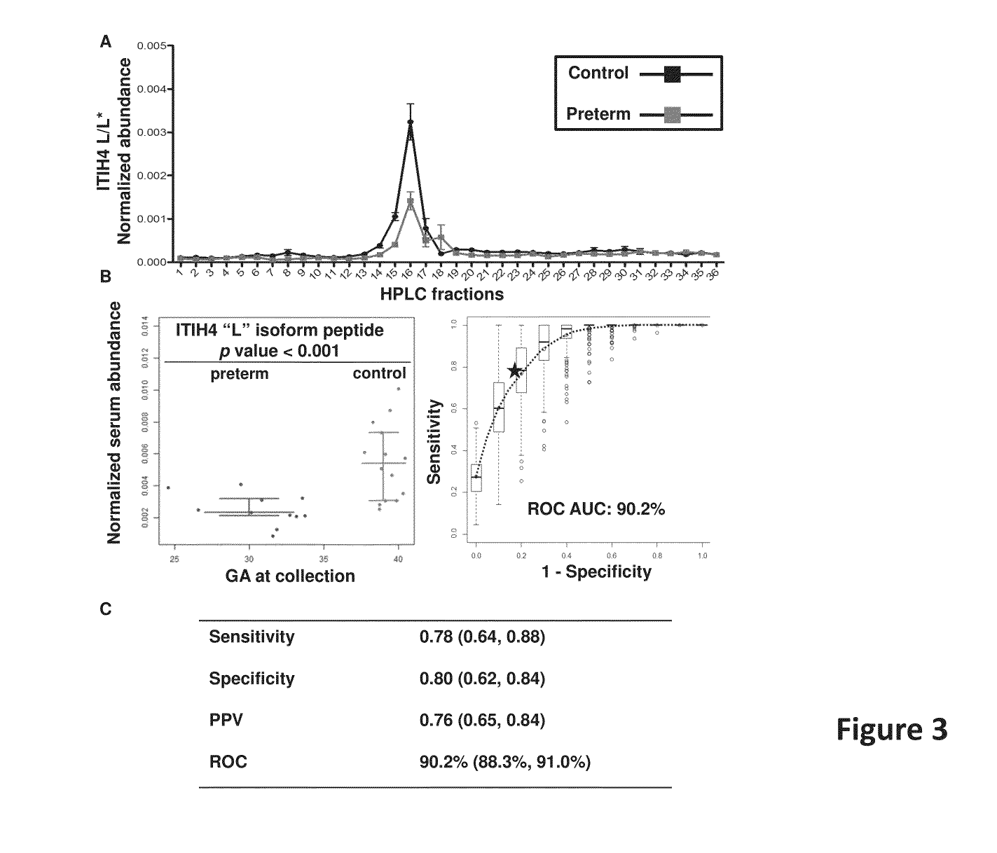

As shown in FIG. 3, the detection of the subject preterm peptide biomarkers at 24 weeks of gestation provides a sensitivity of 78%, a specificity of 80% or better, and an accuracy of 90.2%. The sensitivity, specificity and accuracy of other peptide panels comprising this encompassed herein may be readily determined using the above mathematical formulas.

### Reagents, Devices and Kits

Also provided are reagents, devices and kits thereof for practicing one or more of the above-described methods. The subject reagents, systems and kits thereof may vary greatly. Reagents of interest include reagents specifically designed for use in producing the above-described preterm birth score for a sample, for example, one or more detection elements, e.g. antibodies or mass spec reagents for the detection of the subject preterm peptide biomarkers. In some instances, the detection element comprises reagent(s) to detect one or more peptide markers, for example, the detection element may be a dipstick, a plate, an array, or cocktail that comprises one or more detection elements, e.g. one or more antibodies, which may be used to detect the expression of one or more preterm peptide biomarkers simultaneously,

One type of reagent that is specifically tailored for quantifying peptides in a biological sample is a collection of isotope labeled- and unlabeled-peptides that may be used as quantification reagents, for example for calibration and as internal references, e.g. in spectrometry methods, e.g. mass spectrometry (MS)-based methods.

Another type of reagent that is specifically tailored for quantifying peptides in a biological sample is a collection of antibodies that bind specifically to the preterm peptide biomarkers of interest, e.g. in an ELISA format, in an xMAP™ microsphere format, on a proteomic array, in suspension for analysis by flow cytometry, by western blotting, by dot blotting, or by immunohistochemistry. Usually, the antibodies are specific for the preterm peptide biomarker(s) of interest but not the polypeptide(s) from which they were derived, e.g. the antibody will be specific for QLGLPGPPDVPDHAAYHPF (SEQ ID NO:6) and/or LLGLPGPPDVPDHAAYHPF (SEQ ID NO:5), but not the ITIH4 polypeptide. Typically, such antibodies will be specific for a domain created by the cleavage event that generated the peptide. Antibodies that are specific to the polypeptide(s) and not the peptide marker(s) may also be included, which serve as negative control(s).

In some instances, a system may be provided. As used herein, the term “system” refers to a collection of reagents, however compiled, e.g., by purchasing the collection of reagents from the same or different sources. In some instances, a kit may be provided. As used herein, the term “kit” refers to a collection of reagents provided, e.g., sold, together. For example, the peptide-based detection of the sample may be coupled with data processing platform that will allow multiparameter determination of the subject peptide biomarkers and, in some embodiments, preterm peptide biomarkers known in the art for personalized preterm care.

The systems and kits of the subject invention may include the above-described peptides or peptide-specific antibody collections. The systems and kits may further include one or more additional reagents employed in the various methods, such as liquid chromatography columns, e.g. HPLC columns, for initial purification of the peptides, fractionation vials, etc., various buffer mediums, e.g. hybridization and washing buffers, labeled probe purification reagents and components, like spin columns, etc., signal generation and detection reagents, e.g. labeled secondary antibodies, streptavidin-alkaline phosphatase conjugate, chemifluorescent or chemiluminescent substrate, and the like.

The subject systems and kits may also include a reference, which element is, in many embodiments, a control preterm birth sample or control preterm birth score that can be employed, e.g., by a suitable experimental or computing means, to make a preterm prognosis based on an “input” marker level profile, e.g., that has been determined with the above described reference. Representative references include samples from individuals that ultimately gave birth prematurely, or samples from individuals that gave birth after full term, databases of preterm birth scores e.g., reference or control scores, and the like, as described above.

In addition to the above components, the subject kits will further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, etc., on which the information has been recorded. Yet another means that may be present is a website address which may be used via the internet to access the information at a removed site. Any convenient means may be present in the kits.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference. Reagents, cloning vectors, and kits for genetic manipulation referred to in this disclosure are available from commercial vendors such as BioRad, Stratagene, Invitrogen, Sigma-Aldrich, and ClonTech.

The use of biofluid (e.g. serum or urine) for the analysis of the naturally occurring peptidome (MW <4000) as a source of biomarkers has been reported in different diseases (Ling, X. B. et al. (2010). Urine Peptidomic and Targeted Plasma Protein Analyses in the Diagnosis and Monitoring of Systemic Juvenile Idiopathic Arthritis. Clin Proteomics 6, 175-193; Ling, X. B. et al. (2011). A diagnostic algorithm combining clinical and molecular data distinguishes Kawasaki disease from other febrile illnesses. BMC Med 9, 130; Ling, X. B., Mellins, E. D., Sylvester, K. G. and Cohen, H. J. (2010). Urine peptidomics for clinical biomarker discovery. Advances in clinical chemistry 51, 181-213; Ling, X. B., Sigdel, T. K., Lau, K., Ying, L., Lau, I., Schilling, J. and Sarwal, M. M. (2010). Integrative urinary peptidomics in renal transplantation identifies biomarkers for acute rejection. J Am Soc Nephrol 21, 646-53; Villanueva, J. et al. (2006). Differential exoprotease activities confer tumor-specific serum peptidome patterns. J Clin Invest 116, 271-84). For clinical application, mass spectrometry-based profiling of naturally occurring peptides can provide an extensive inventory of serum peptides derived from either high-abundant endogenous circulating proteins or cell and tissue proteins (Liotta, L. A. and Petricoin, E. F. (2006). Serum peptidome for cancer detection: spinning biologic trash into diagnostic gold. J Clin Invest 116, 26-30). These peptides are usually soluble, stable from endogenous proteases or peptidases, and can be directly used for liquid chromatography-mass spectrometry (LC/MS) analysis without additional manipulation (e.g. tryptic digests).

A previous study by Esplin et al. attempted to use serum peptidomic patterns to identify patients with preterm birth (Esplin, M. S. et al. (2011). Proteomic identification of serum peptides predicting subsequent spontaneous preterm birth. Am J Obstet Gynecol 204, 391 e1-8). An inter-alpha-trypsin inhibitor heavy chain 4 (ITIH4) peptide (QLGLPGPPDVPDHAAYHPF) was identified as a preterm birth biomarker from a predominantly (˜75%) African-American cohort. The serum level of ITIH4 peptide biomarker was shown to decrease in women who delivered preterm with a sensitivity of 65.0% and specificity of 82.5% for discriminating preterm delivery from term pregnancies. The biological activity of the parent protein or the fragment identified remained unknown.

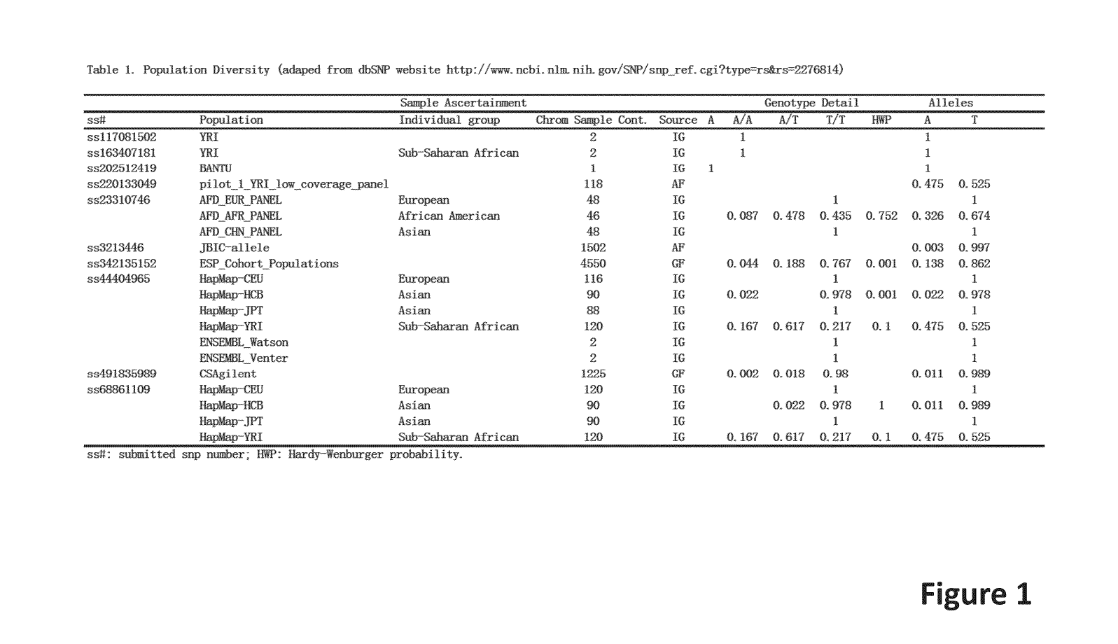

The NCBI SNP database of common gene variations indicates that there is a single nucleotide polymorphism (SNP, variant rs2276814) in ITIH4 (position 669) where a single coding nucleotide differs from A of amino acid codon cAa to T of cTa, resulting in an amino acid change from glutamine (Q) to leucine (L). Shown in FIG. 1, African American or Sub-Saharan African subjects have comparable probabilities of “A” or “T” allele, and therefore, with similar chances of glutamine (Q) or leucine (L) at ITIH4 position 669. In contrast, European and Asian subjects are predominantly homozygous for the “T” allele, and therefore, with leucine at position 669 of ITIH4. Within the European and Asian populations, it is expected the ITIH4 derived serum peptide should be in its single nucleotide polymorphic isoform (protein ITIH4 669, nucleotide level A→T, protein level Q→L). This “L” isoform peptide sequence should be LLGLPGPPDVPDHAAYHPF (SEQ ID NO:5), which shares an almost identical sequence as the preterm birth serum peptide biomarker “Q” isoform (QLGLPGPPDVPDHAAYHPF (SEQ ID NO:6)) but the first amino acid in the sequence is changed from L to Q.

LCMS based quantitative proteomics analysis is a powerful method for selective quantification of specific proteins/peptides in very complex mixtures. LCMS method coupled with stable isotope dilution (SID) provides both absolute structural specificity for the analyte and relative or absolute measurement of analyte concentration (Addona, T. A. et al. (2009). Multi-site assessment of the precision and reproducibility of multiple reaction monitoring-based measurements of proteins in plasma. Nat Biotechnol 27, 633-41). Unlike the label-free quantitative method, which is more error-prone due to systematic variations among individual runs and the stochastic nature of indices used for calculation (Kito, K. and Ito, T. (2008). Mass spectrometry-based approaches toward absolute quantitative proteomics. Curr Genomics 9, 263-74), the SID based peptide assay method remains the gold standard for absolute protein quantitation via mass spectrometry. Targeted mass spectrometry (also known as data-dependent acquisition), if focusing on “Q” isoform's specific mass/charge (m/z) ratio and at specific liquid chromatography time as reported previously (Esplin, M. S. et al. (2011), supra), would fail to detect the readout of the ITIH “L” isoform peptide.

Quantitative proteomics are used below to demonstrate that the ITIH4 “L” isoform peptide could be detected in sera of pregnant women of Asian, European, or mixed background (e.g. Hispanics), and that this “L” isoform is a biomarker associated with preterm birth in these women.

### Materials and Methods

Specimen collection and preprocessing. To qualify the ITIH4 peptide serum “L” isoform as a preterm birth biomarker, we selected 11 case women who delivered preterm (before 37 weeks gestation) and 14 control women who delivered at term and matched on age, ethnicity, and maternal labor (Table 1). Serum samples were prospectively collected from women at the time of labor presentation, i.e. 24-34 weeks gestation and subsequent preterm delivery, or at 37-42 weeks and subsequent term delivery. 25 μL aliquot of patient serum was mixed with 75 μL of 100% methanol, and was vortex mixed for about 30 minutes. Supernatants were transferred into a 96 well plate after centrifugation at 3000 rpm (˜1,700 g) for 10 minutes. The extracted samples' peptide concentrations were quantified by 2,4,6-Trinitrobenzene Sulfonic Acid (TNBSA or TNBS) kit (TS-28997, Thermo Fisher, Calif., USA).

TABLE 1
Characteristics of the patients used in the study.

| | Case | Control | p value |
|---|---|---|---|
| Characteristic | (n = 11) | (n = 14) | Case vs. Control |
| Age | | | 0.811 |
| Mean | 29.1 | 28.5 | |
| (SD) | (4.9) | (6.8) | |
| Race | | | 0.451 |
| African-American | 1 (9.1%) | 0 (0%) | |
| Asian | 3 (27.3%) | 2 (14.3%) | |
| Caucasian | 2 (18.2%) | 1 (7.1%) | |
| Hispanic | 5 (45.5%) | 10 (71.4%) | |
| Pacific Islander | 0 (0%) | 1 (7.1%) | |
| Gestation age @ | | | <0.001 |
| delivery (week) | | | |
| Mean | 32.74 | 39.30 | |
| (SD) | (3.32) | (0.94) | |
| Gestation age @ | | | <0.001 |
| collection (week) | | | |
| Mean | 30.74 | 39.21 | |
| (SD) | (2.93) | (0.77) | |
| Gap between | | | <0.001 |
| collection and Delivery | | | |
| Mean | 2 | 0.08 | |
| (SD) | (2.58) | (0.50) | |
| Birth weight | | | <0.001 |
| (grams) | | | |
| Mean | 2069.9 | 3533.9 | |
| (SD) | (663.7) | (306.1) | |
| Labor | | | 0.288 |
| Yes | 8 (72.7%) | 13(92.9%) | |

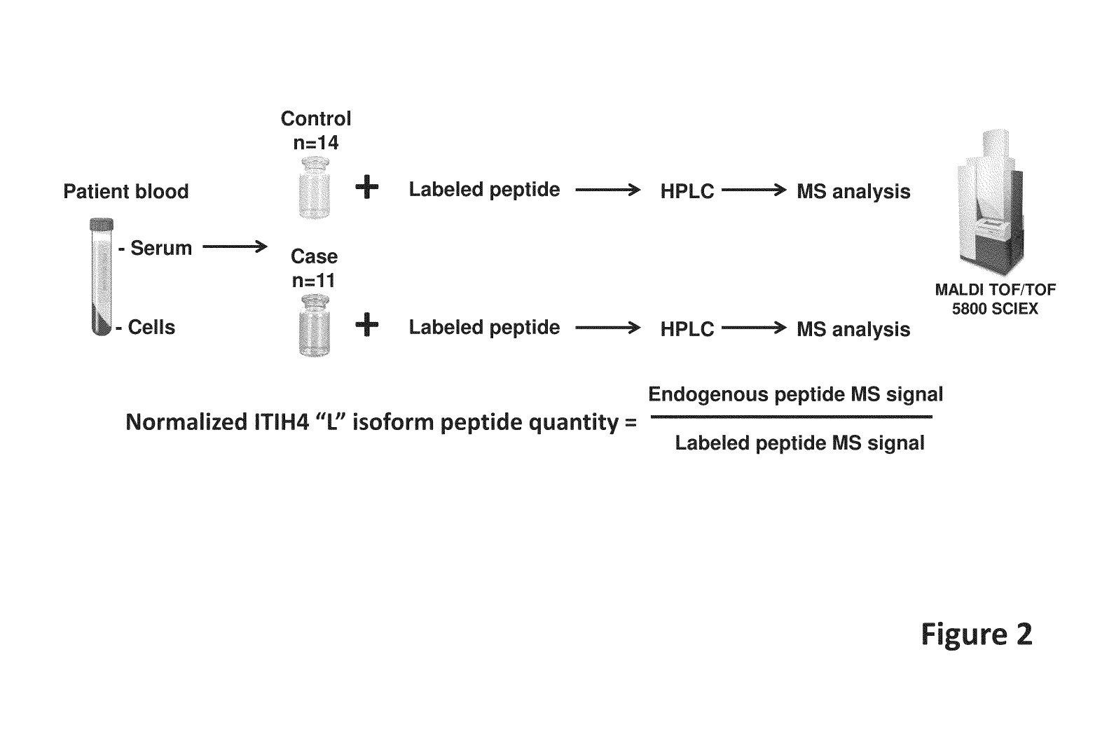

Mass spectrometric quantification of the serum ITIH4 “L” isoform peptide. For absolute quantification method using stable isotope labeled synthetic marker analogues, we chose the stable isotope labeled (with five 13C-labeled and one 15N-labeled for each proline) “L” isoform ITIH4 peptide synthesized by AnaSpec Inc. (CA, USA). Therefore, the synthetic labeled peptide had a total mass difference of 30 atomic mass units from the endogenous serum peptide. As shown in FIG. 2, stable isotope labeled peptide was added as a quantification reference in defined amounts to the serum peptide samples prior to the liquid chromatography (C18 column) mass spectrometric analysis using AB15800 matrix-assisted laser desorption/ionization (MALDI) TOF (Time of Flight). Each sample's endogenous ITIH4 “L” isoform peptide abundance was normalized to the isotope reference peptide for subsequent statistical analysis.

Statistical analyses. Odds ratio and its confidence interval were calculated using generalized linear regression modeling (GLM). The diagnostic performance of the ITIH4 “L” isoform peptide was evaluated by ROC curve analysis (Zweig, M. H. and Campbell, G. (1993). Receiver-operating characteristic (ROC) plots: a fundamental evaluation tool in clinical medicine. Clin Chem 39, 561-77; Sing, T., Sander, 0., Beerenwinkel, N. and Lengauer, T. (2005). ROCR: visualizing classifier performance in R. Bioinformatics 21, 3940-1). The “cut point” along the ROC curve was determined as previously described (Zweig, M. H. and Campbell, G. (1993), supra) to obtain the optimal sensitivity and specificity of the assay.

### Results

Summarized in FIG. 3A, the peaks in the chromatogram were formed by the elution of “L” isoform peptide from C18 column at the 16th time point. The normalized “L” isoform peptide serum concentration in case samples was found to decrease 3-fold compared to controls. Scatter plot analysis (FIG. 3B left panel) revealed that the ITIH4 “L” isoform was significantly lower (both Student T test and Mann-Whitney U test, p value<0.001) in mothers' delivering preterm babies compared to those women delivering term babies. Thus, indicating that ITIH4 “L” isoform serum peptide can be detected in a cohort predominantly of Asian and European origin; and reduced serum levels of ITIH4 “L” isoform is associated with preterm birth. The ROC AUC was estimated as 90.2%. The cut point analysis (FIG. 3C) showed that ITIH4 “L” peptide based assay could be optimized with sensitivity (0.78) and specificity (0.80).

### Discussion

Our results of serum levels of “L” isoform ITIH4 peptide in predominantly Hispanic, Asian and Caucasian women are consistent with the SNP database allele frequency records, i.e., subjects of Asian or European origin are largely of “L” at ITIH4 669 (Table 1), and often lack the ITIH4 “Q” isoform. Therefore, clinical application of the African American cohort derived analytics, which associated the marked reduction of serum ITIH4 “Q” isoform with preterm birth risk, would not be applicable to women of other ethnic origins. Targeted analysis of ITIH4 “Q” isoform alone as previously described would be insufficient for preterm birth risk profiling of patients of diverse race-ethnic backgrounds.

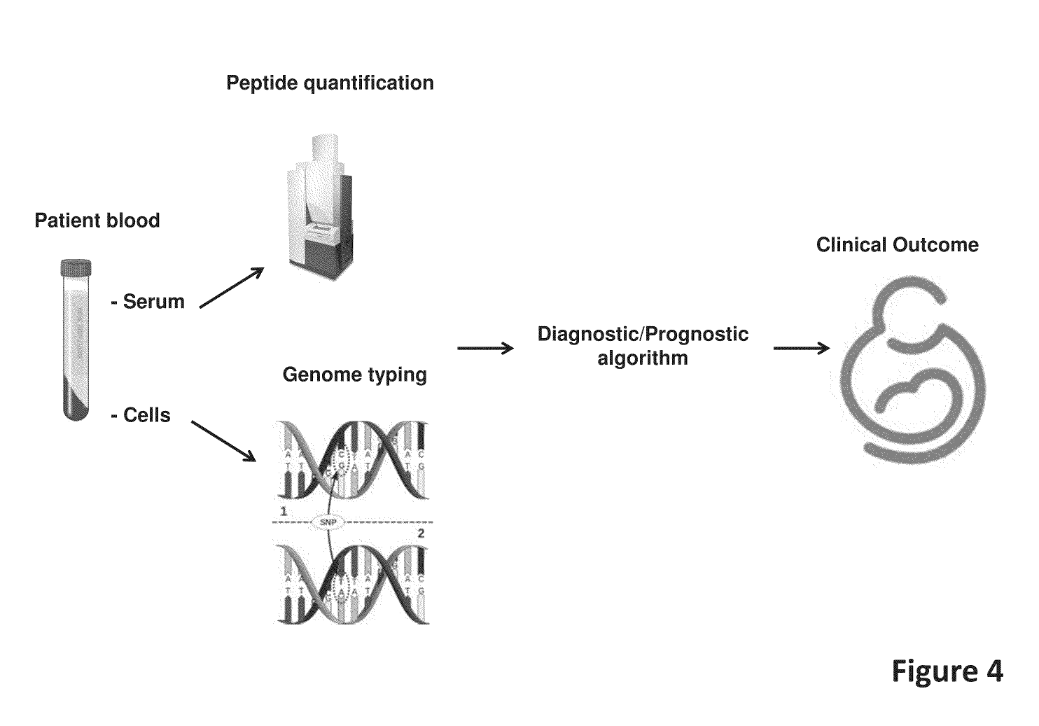

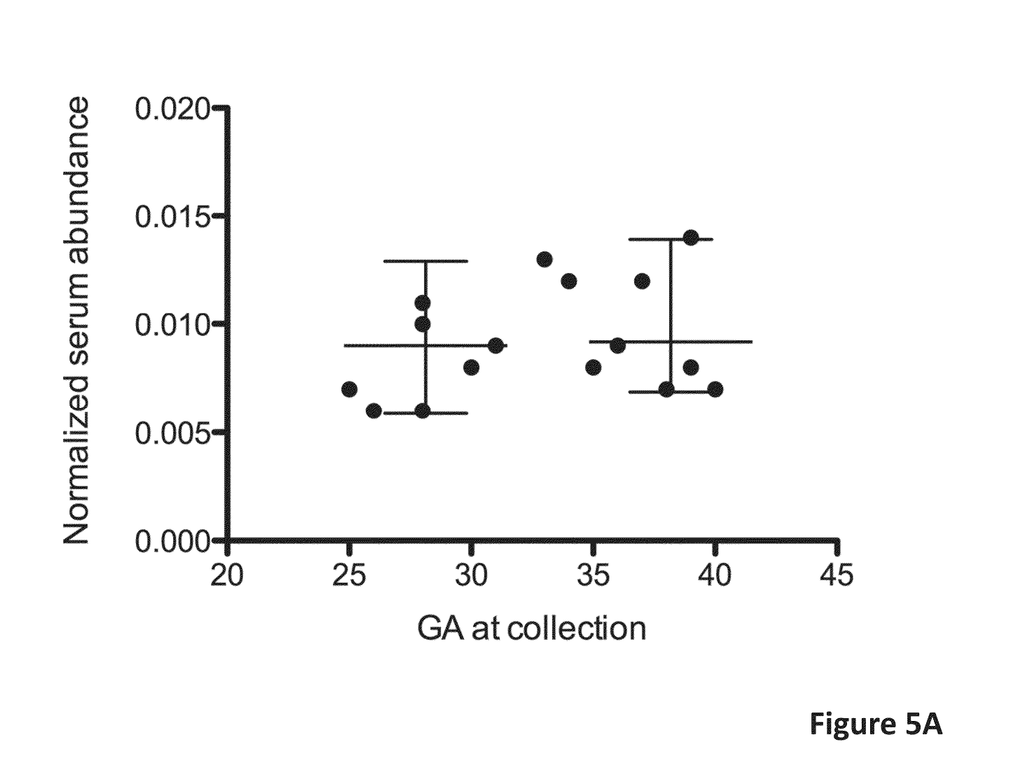

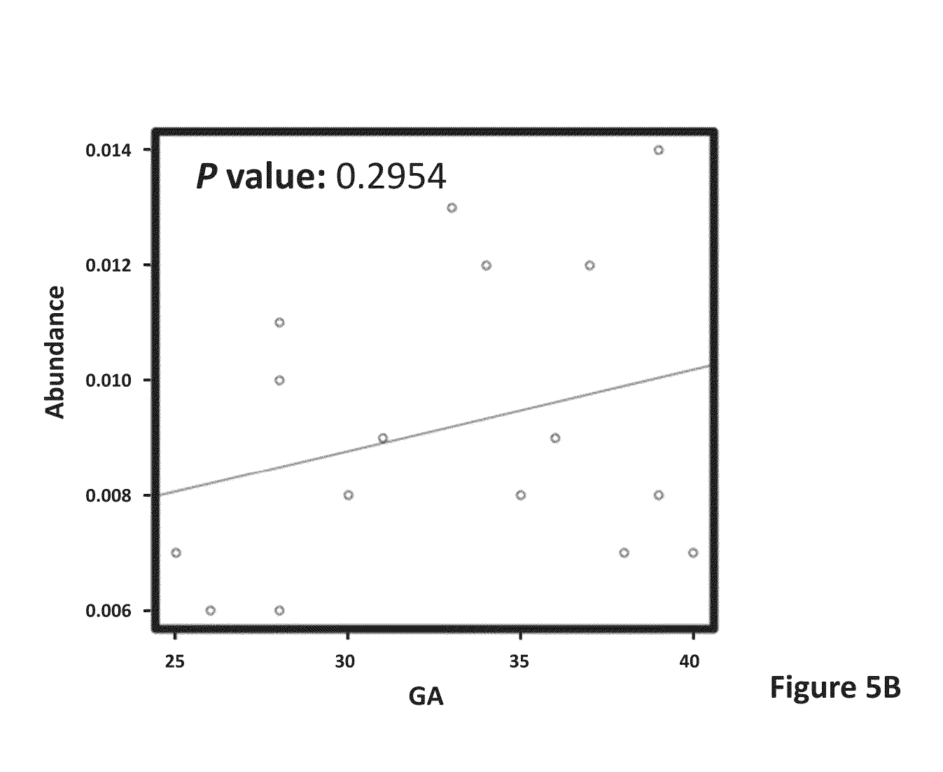

Thus, our results (using the ITIH4 “L” isoform) demonstrate the importance of accounting for the race-ethnic variation when applying the ITIH4 serum peptide as a predictive biomarker for preterm birth. To comprehensively apply the ITIH4 serum peptide biomarker for preterm birth analysis in all ethnic backgrounds, we propose a multiple-stage algorithm (FIG. 4). At the first stage, blood cells will be processed and SNP genotyping will be performed to determine whether the subject's ITIH4 669 allele is in “Q” or “L” isoform. At the second stage, upon the genotyping results, targeted analysis of either ITIH4 “Q” (homozygous genome typing), “L” (homozygous genome typing), or sum of the two (heterozygous genome typing) in the patient serum will be conducted. At the final stage, the serum quantity of ITIH4 peptide isoform(s) would be used, in combination with other protein markers (Esplin, M. S. et al. (2011). Proteomic identification of serum peptides predicting subsequent spontaneous preterm birth. Am J Obstet Gynecol 204, 391 e1-8), to estimate a patient's risk for preterm birth.

Our examination of the ITIH4 “L” isoform as a biomarker for preterm birth is based on a small number of case and control women and the specimens derive from different gestational age between cases and controls. We acknowledge these as limitations. Depite these limitations it is still noteworthy that subjects of Asian or European origin are largely of ITIH4 “L” isoform, and often lack the ITIH4 “Q” isoform. Furthermore, the decreased abundance of ITIH4 “L” isoform in preterm birth patients is the same as “Q” isoform reported by Esplin et.al. Our novel findings need to be replicated in a larger group of case and control women with harmonized serum collection points during gestation.

### Conclusion

We believe this association of the ITIH4 “L” isoform with preterm birth complements the previous findings of the “Q” isoform (Esplin, M. S. et al. (2011). Proteomic identification of serum peptides predicting subsequent spontaneous preterm birth. Am J Obstet Gynecol 204, 391 e1-8) as a preterm birth biomarker. Future prospective trial of our proposed multiple-stage algorithm (FIG. 4: ITIH4 669 SNP allele typing plus the targeted serum ITIH4 peptide analysis), may lead to a clinically applicable test predicting preterm birth in patients of diverse ethnic backgrounds.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of the present invention is embodied by the appended claims.

That which is claimed is:

1. A method for providing a preterm birth prognosis for a pregnant individual, monitoring a pregnant individual for increased risked of preterm birth, or making a treatment decision for a pregnant individual, the method comprising:
obtaining a determination of the amount of preterm peptide biomarker LLGLPGPPDVPDHAAYHPF (SEQ ID NO:5) in a biological sample from the pregnant individual, and
providing a preterm birth prognosis for the individual, monitoring a pregnant individual for increased risked of preterm birth, or making a treatment decision for the pregnant individual based on the amount determined.

2. The method according to claim 1, wherein the determination of the amount comprises measuring the amount of preterm peptide biomarker in the biological sample by mass spectrometry.

3. The method according to claim 2, wherein the selecting comprises genotyping the individual for an rs2276814 polymorphism, and selecting the preterm peptide biomarker LLGLPGPPDVPDHAAYHPF (SEQ ID NO:5) based on the genotype.

4. The method according to claim 2, wherein the selecting comprises performing a demographic survey of the ethnicity of the individual, and selecting the preterm peptide biomarker based on the demographic survey.

5. A method for selecting an ITIH4 preterm peptide biomarker for a pregnant individual, the method comprising:
obtaining a rs2276814 polymorphism result or a demographic survey of ethnicity result for a pregnant individual, and
selecting, based on the rs2276814 polymorphism result or demographic survey result, a preterm peptide biomarker for the individual.

6. The method according to claim 5, wherein the obtaining a rs2276814 polymorphism result comprises genotyping the individual for the rs2276814,
wherein, if the individual comprises an A allele at rs2276814, the preterm peptide biomarker QLGLPGPPDVPDHAAYHPF (SEQ ID NO:6) is selected, and if the individual comprises a T allele at rs2276814, the preterm peptide biomarker LLGLPGPPDVPDHAAYHPF (SEQ ID NO:5) is selected.

7. The method according to claim 6, wherein the obtaining a demographic survey of ethnicity result comprises asking if the ethnicity of the individual is African American, Caucasian, or Hispanic;
wherein, if the ethnicity of the individual is African American, the preterm peptide biomarker QLGLPGPPDVPDHAAYHPF (SEQ ID NO:6) is selected, and if the ethnicity of the individual is Caucasian or Hispanic, the preterm peptide biomarker LLGLPGPPDVPDHAAYHPF (SEQ ID NO:5) is selected.

8. A method for providing a preterm birth prognosis for a pregnant individual, monitoring a pregnant individual for increased risked of preterm birth, or making a treatment decision for a pregnant individual, the method comprising:
selecting a preterm peptide biomarker from the group consisting of LLGLPGPPDVPDHAAYHPF (SEQ ID NO:5) and QLGLPGPPDVPDHAAYHPF (SEQ ID NO:6);
obtaining, based on the selection, a determination of the amount of peptide LLGLPGPPDVPDHAAYHPF (SEQ ID NO:5) or QLGLPGPPDVPDHAAYHPF (SEQ ID NO:6) in a biological sample from the pregnant individual; and
providing a preterm birth prognosis for the individual, monitoring a pregnant individual for increased risked of preterm birth, or making a treatment decision for the pregnant individual based on the amount determined.

9. The method according to claim 8, wherein the obtaining a rs2276814 polymorphism result comprises genotyping the individual for the rs2276814,
wherein, if the individual comprises an A allele at rs2276814, the preterm peptide biomarker QLGLPGPPDVPDHAAYHPF (SEQ ID NO:6) is selected, and if the individual comprises a T allele at rs2276814, the preterm peptide biomarker LLGLPGPPDVPDHAAYHPF (SEQ ID NO:5) is selected.

10. The method according to claim 8, wherein the obtaining a demographic survey of ethnicity result comprises asking if the ethnicity of the individual is African American, Caucasian, or Hispanic;
Wherein, if the ethnicity of the individual is African American, the preterm peptide biomarker QLGLPGPPDVPDHAAYHPF (SEQ ID NO:6) is selected, and if the ethnicity of the individual is Caucasian or Hispanic, the preterm peptide biomarker LLGLPGPPDVPDHAAYHPF (SEQ ID NO:5) is selected.

11. The method according to claim 10, wherein the determination of the amount comprises measuring the amount of preterm peptide biomarker in the biological sample by mass spectrometry.

12. A kit for determining the risk of a preterm birth, comprising:
a rs2276814 polymorphism detection agent, and
an ITIH4 preterm peptide biomarker detection quantification reagent.

13. The kit according to claim 11, wherein the ITIH4 preterm peptide biomarker quantification reagent is an LLGLPGPPDVPDHAAYHPF (SEQ ID NO:5) isotope labeled-peptide.


SEQUENCE LISTING (8 sequences)

SEQ ID NO: 1 - DNA, 3283 bp, Homo sapiens
taatccattccccacttgctgtcgagttcagaagcctcctggcagacactggagccacgatgaagcccccaaggcctgtccgtacctgcagcaaagttctcgtcctgctttcactgctggccatccaccagactactactgccgaaaagaatggcatcgacatctacagcctcaccgtggactccagggtctcatcccgatttgcccacacggtcgtcaccagccgagtggtcaatagggccaatactgtgcaggaggccaccttccagatggagctgcccaagaaagccttcatcaccaacttctccatgatcatcgatggcatgacctacccagggatcatcaaggagaaggctgaagcccaggcacagtacagcgcagcagtggccaagggaaagagcgctggcctcgtcaaggccaccgggagaaacatggagcagttccaggtgtcggtcagtgtggctcccaatgccaagatcacctttgagctggtctatgaggagctgctcaagcggcgtttgggggtgtacgagctgctgctgaaagtgcggccccagcagctggtcaagcacctgcagatggacattcacatcttcgagccccagggcatcagctttctggagacagagagcaccttcatgaccaaccagctggtagacgccctcaccacctggcagaataagaccaaggctcacatccggttcaagccaacactttcccagcagcaaaagtccccagagcagcaagaaacagtcctggacggcaacctcattatccgctatgatgtggaccgggccatctccgggggctccattcagatcgagaacggctactttgtacactactttgcccccgagggcctaaccacaatgcccaagaatgtggtctttgtcattgacaagagcggctccatgagtggcaggaaaatccagcagacccgggaagccctaatcaagatcctggatgacctcagccccagagaccagttcaacctcatcgtcttcagtacagaagcaactcagtggaggccatcactggtgccagcctcagccgagaacgtgaacaaggccaggagctttgctgcgggcatccaggccctgggagggaccaacatcaatgatgcaatgctgatggctgtgcagttgctggacagcagcaaccaggaggagcggctgcccgaagggagtgtctcactcatcatcctgctcaccgatggcgaccccactgtgggggagactaaccccaggagcatccagaataacgtgcgggaagctgtaagtggccggtacagcctcttctgcctgggcttcggtttcgacgtcagctatgccttcctggagaagctggcactggacaatggcggcctggcccggcgcatccatgaggactcagactctgccctgcagctccaggacttctaccaggaagtggccaacccactgctgacagcagtgaccttcgagtacccaagcaatgccgtggaggaggtcactcagaacaacttccggctcctcttcaagggctcagagatggtggtggctgggaagctccaggaccgggggcctgatgtgctcacagccacagtcagtgggaagctgcctacacagaacatcactttccaaacggagtccagtgtggcagagcaggaggcggagttccagagccccaagtatatcttccacaacttcatggagaggctctgggcatacctgactatccagcagctgctggagcaaactgtctccgcatccgatgctgatcagcaggccctccggaaccaagcgctgaatttatcacttgcctacagctttgtcacgcctctcacatctatggtagtcaccaaacccgatgaccaagagcagtctcaagttgctgagaagcccatggaaggcgaaagtagaaacaggaatgtccactcaggttccactttcttcaaatattatctccagggagcaaaaataccaaaaccagaggcttccttttctccaagaagaggatggaatagacaagctggagctgctggctcccggatgaatttcagacctggggttctcagctccaggcaacttggactcccaggacctcctgatgttcctgaccatgctgcttaccaccccttccgccgtctggccatcttgcctgcttcagcaccaccagccacctcaaatcctgatccagctgtgtctcgtgtcatgaatatgaaaatcgaagaaacaaccatgacaacccaaaccccagcccccatacaggctccctctgccatcctgccactgcctgggcagagtgtggagcggctctgtgtggaccccagacaccgccaggggccagtgaacctgctctcagaccctgagcaaggggttgaggtgactggccagtatgagagggagaaggctgggttctcatggatcgaagtgaccttcaagaaccccctggtatgggttcacgcatcccctgaacacgtggtggtgactcggaaccgaagaagctctgcgtacaagtggaaggagacgctattctcagtgatgcccggcctgaagatgaccatggacaagacgggtctcctgctgctcagtgacccagacaaagtgaccatcggcctgttgttctgggatggccgtggggaggggctccggctccttctgcgtgacactgaccgcttctccagccacgttggagggacccttggccagttttaccaggaggtgctctggggatctccagcagcatcagatgacggcagacgcacgctgagggttcagggcaatgaccactctgccaccagagagcgcaggctggattaccaggaggggcccccgggagtggagatttcctgctggtctgtggagctgtagttctgatggaaggagctgtgcccaccctgtacacttggcttccccctgcaactgcagggccgcttctggggcctggaccaccatggggaggaagagtcccactcattacaaataaagaaaggtggtgtgagcctgggaagtgggtgtctccagttccatgtggccaaatcctagggcctcaacctcgcatcctgaaccttagcatcgtggaacacagaagcttccactgtcagctctcaagagcccatggccaggaaggcccatgctgagctttcagtccagccccttcattttacaaacaaggaaactgagctcgaaccacccatttgagatgtcactgtggcccccagctagaggcccagggctgggagcattctccaggagcagaggttcagtctgcttcatggtctcttggaccagttttgactaca

SEQ ID NO: 2 - protein, 930 aa, Homo sapiens
MKPPRPVRTCSKVLVLLSLLAIHQTTTAEKNGIDIYSLTVDSRVSSRFAHTVVTSRVVNRANTVQEATFQMELPKKAFITNFSMIIDGMTYPGIIKEKAEAQAQYSAAVAKGKSAGLVKATGRNMEQFQVSVSVAPNAKITFELVYEELLKRRLGVYELLLKVRPQQLVKHLQMDIHIFEPQGISFLETESTFMTNQLVDALTTWQNKTKAHIRFKPTLSQQQKSPEQQETVLDGNLIIRYDVDRAISGGSIQIENGYFVHYFAPEGLTTMPKNVVFVIDKSGSMSGRKIQQTREALIKILDDLSPRDQFNLIVFSTEATQWRPSLVPASAENVNKARSFAAGIQALGGTNINDAMLMAVQLLDSSNQEERLPEGSVSLIILLTDGDPTVGETNPRSIQNNVREAVSGRYSLFCLGFGFDVSYAFLEKLALDNGGLARRIHEDSDSALQLQDFYQEVANPLLTAVTFEYPSNAVEEVTQNNFRLLFKGSEMVVAGKLQDRGPDVLTATVSGKLPTQNITFQTESSVAEQEAEFQSPKYIFHNFMERLWAYLTIQQLLEQTVSASDADQQALRNQALNLSLAYSFVTPLTSMVVTKPDDQEQSQVAEKPMEGESRNRNVHSGSTFFKYYLQGAKIPKPEASFSPRRGWNRQAGAAGSRMNFRPGVLSSRQLGLPGPPDVPDHAAYHPFRRLAILPASAPPATSNPDPAVSRVMNMKIEETTMTTQTPAPIQAPSAILPLPGQSVERLCVDPRHRQGPVNLLSDPEQGVEVTGQYEREKAGFSWIEVTFKNPLVWVHASPEHVVVTRNRRSSAYKWKETLFSVMPGLKMTMDKTGLLLLSDPDKVTIGLLFWDGRGEGLRLLLRDTDRFSSHVGGTLGQFYQEVLWGSPAASDDGRRTLRVQGNDHSATRERRLDYQEGPPGVEISCWSVEL

SEQ ID NO: 3 - DNA, 3193 bp, Homo sapiens
taatccattccccacttgctgtcgagttcagaagcctcctggcagacactggagccacgatgaagcccccaaggcctgtccgtacctgcagcaaagttctcgtcctgctttcactgctggccatccaccagactactactgccgaaaagaatggcatcgacatctacagcctcaccgtggactccagggtctcatcccgatttgcccacacggtcgtcaccagccgagtggtcaatagggccaatactgtgcaggaggccaccttccagatggagctgcccaagaaagccttcatcaccaacttctccatgatcatcgatggcatgacctacccagggatcatcaaggagaaggctgaagcccaggcacagtacagcgcagcagtggccaagggaaagagcgctggcctcgtcaaggccaccgggagaaacatggagcagttccaggtgtcggtcagtgtggctcccaatgccaagatcacctttgagctggtctatgaggagctgctcaagcggcgtttgggggtgtacgagctgctgctgaaagtgcggccccagcagctggtcaagcacctgcagatggacattcacatcttcgagccccagggcatcagctttctggagacagagagcaccttcatgaccaaccagctggtagacgccctcaccacctggcagaataagaccaaggctcacatccggttcaagccaacactttcccagcagcaaaagtccccagagcagcaagaaacagtcctggacggcaacctcattatccgctatgatgtggaccgggccatctccgggggctccattcagatcgagaacggctactttgtacactactttgcccccgagggcctaaccacaatgcccaagaatgtggtctttgtcattgacaagagcggctccatgagtggcaggaaaatccagcagacccgggaagccctaatcaagatcctggatgacctcagccccagagaccagttcaacctcatcgtcttcagtacagaagcaactcagtggaggccatcactggtgccagcctcagccgagaacgtgaacaaggccaggagctttgctgcgggcatccaggccctgggagggaccaacatcaatgatgcaatgctgatggctgtgcagttgctggacagcagcaaccaggaggagcggctgcccgaagggagtgtctcactcatcatcctgctcaccgatggcgaccccactgtgggggagactaaccccaggagcatccagaataacgtgcgggaagctgtaagtggccggtacagcctcttctgcctgggcttcggtttcgacgtcagctatgccttcctggagaagctggcactggacaatggcggcctggcccggcgcatccatgaggactcagactctgccctgcagctccaggacttctaccaggaagtggccaacccactgctgacagcagtgaccttcgagtacccaagcaatgccgtggaggaggtcactcagaacaacttccggctcctcttcaagggctcagagatggtggtggctgggaagctccaggaccgggggcctgatgtgctcacagccacagtcagtgggaagctgcctacacagaacatcactttccaaacggagtccagtgtggcagagcaggaggcggagttccagagccccaagtatatcttccacaacttcatggagaggctctgggcatacctgactatccagcagctgctggagcaaactgtctccgcatccgatgctgatcagcaggccctccggaaccaagcgctgaatttatcacttgcctacagctttgtcacgcctctcacatctatggtagtcaccaaacccgatgaccaagagcagtctcaagttgctgagaagcccatggaaggcgaaagtagaaacaggaatgtccactcagctggagctgctggctcccggatgaatttcagacctggggttctcagctccaggcaacttggactcccaggacctcctgatgttcctgaccatgctgcttaccaccccttccgccgtctggccatcttgcctgcttcagcaccaccagccacctcaaatcctgatccagctgtgtctcgtgtcatgaatatgaaaatcgaagaaacaaccatgacaacccaaaccccagcccccatacaggctccctctgccatcctgccactgcctgggcagagtgtggagcggctctgtgtggaccccagacaccgccaggggccagtgaacctgctctcagaccctgagcaaggggttgaggtgactggccagtatgagagggagaaggctgggttctcatggatcgaagtgaccttcaagaaccccctggtatgggttcacgcatcccctgaacacgtggtggtgactcggaaccgaagaagctctgcgtacaagtggaaggagacgctattctcagtgatgcccggcctgaagatgaccatggacaagacgggtctcctgctgctcagtgacccagacaaagtgaccatcggcctgttgttctgggatggccgtggggaggggctccggctccttctgcgtgacactgaccgcttctccagccacgttggagggacccttggccagttttaccaggaggtgctctggggatctccagcagcatcagatgacggcagacgcacgctgagggttcagggcaatgaccactctgccaccagagagcgcaggctggattaccaggaggggcccccgggagtggagatttcctgctggtctgtggagctgtagttctgatggaaggagctgtgcccaccctgtacacttggcttccccctgcaactgcagggccgcttctggggcctggaccaccatggggaggaagagtcccactcattacaaataaagaaaggtggtgtgagcctgggaagtgggtgtctccagttccatgtggccaaatcctagggcctcaacctcgcatcctgaaccttagcatcgtggaacacagaagcttccactgtcagctctcaagagcccatggccaggaaggcccatgctgagctttcagtccagccccttcattttacaaacaaggaaactgagctcgaaccacccatttgagatgtcactgtggcccccagctagaggcccagggctgggagcattctccaggagcagaggttcagtctgcttcatggtctcttggaccagttttgactaca

SEQ ID NO: 4 - protein, 900 aa, Homo sapiens
MKPPRPVRTCSKVLVLLSLLAIHQTTTAEKNGIDIYSLTVDSRVSSRFAHTVVTSRVVNRANTVQEATFQMELPKKAFITNFSMIIDGMTYPGIIKEKAEAQAQYSAAVAKGKSAGLVKATGRNMEQFQVSVSVAPNAKITFELVYEELLKRRLGVYELLLKVRPQQLVKHLQMDIHIFEPQGISFLETESTFMTNQLVDALTTWQNKTKAHIRFKPTLSQQQKSPEQQETVLDGNLIIRYDVDRAISGGSIQIENGYFVHYFAPEGLTTMPKNVVFVIDKSGSMSGRKIQQTREALIKILDDLSPRDQFNLIVFSTEATQWRPSLVPASAENVNKARSFAAGIQALGGTNINDAMLMAVQLLDSSNQEERLPEGSVSLIILLTDGDPTVGETNPRSIQNNVREAVSGRYSLFCLGFGFDVSYAFLEKLALDNGGLARRIHEDSDSALQLQDFYQEVANPLLTAVTFEYPSNAVEEVTQNNFRLLFKGSEMVVAGKLQDRGPDVLTATVSGKLPTQNITFQTESSVAEQEAEFQSPKYIFHNFMERLWAYLTIQQLLEQTVSASDADQQALRNQALNLSLAYSFVTPLTSMVVTKPDDQEQSQVAEKPMEGESRNRNVHSAGAAGSRMNFRPGVLSSRQLGLPGPPDVPDHAAYHPFRRLAILPASAPPATSNPDPAVSRVMNMKIEETTMTTQTPAPIQAPSAILPLPGQSVERLCVDPRHRQGPVNLLSDPEQGVEVTGQYEREKAGFSWIEVTFKNPLVWVHASPEHVVVTRNRRSSAYKWKETLFSVMPGLKMTMDKTGLLLLSDPDKVTIGLLFWDGRGEGLRLLLRDTDRFSSHVGGTLGQFYQEVLWGSPAASDDGRRTLRVQGNDHSATRERRLDYQEGPPGVEISCWSVEL

SEQ ID NO: 5 - protein, 19 aa, Homo sapiens
LLGLPGPPDVPDHAAYHPF

SEQ ID NO: 6 - protein, 19 aa, Homo sapines
QLGLPGPPDVPDHAAYHPF

SEQ ID NO: 7 - protein, 41 aa, Homo sapiens
NVHSAGAAGSRMNFRPGVLSSRQLGLPGPPDVPDHAAYHPF

SEQ ID NO: 8 - protein, 28 aa, Homo sapiens
NVHSGSTFFKYYLQGAKIPKPEASFSPR